(19) United States
(12) Plant Patent Application Publication
(10) Pub. No.: US 2023/0116915 P1
(43) Pub. Date: Apr. 13, 2023

(54) LAVANDULA PLANT NAMED 'DC014455LS'

(50) ;

(71) Applicant: LUDO DECOCK, COMINES (BE)

(72) Inventor: LUDO DECOCK, COMINES (BE)

(21) Appl. No.: 17/958,795

(22) Filed: Oct. 3, 2022

Related U.S. Application Data
(60) Provisional application No. 63/252,019, filed on Oct. 4, 2021.

(51) Int. Cl. A01H 6/50

(52) U.S. Cl. PLT/445

(57) ABSTRACT

A new and distinct cultivar of Lavandula plant named ‘DC014455LS’, characterized by its compact, upright and uniformly rounded plant habit; moderately vigorous growth habit; freely branching growth habit; early and freely flowering habit; dark purple-colored flowers and violet-colored terminal flower bracts positioned on strong and erect peduncles; long flowering period; suitability for low temperature production during the winter and early spring; and good garden performance.

### CROSS-REFERENCED TO CLOSELY-RELATED APPLICATIONS & CLAIM FOR PRIORITY TO PROVISIONAL U.S. PATENT APPLICATION

Title: Lavandula Plant Named ‘DC014455LS’

Inventor/Applicant: Ludo Decock

Filed: Oct. 4, 2021

Ser. No.: 63/252,019

Inventor/Applicant hereby claims priority to this provisional U.S. Patent Application.

Botanical designation: Lavandula stoechas.

Cultivar denomination: ‘DC014455LS’.

An European Plant Breeder's Rights application for the instant plant was filed on Jul. 13, 2021, application number 2021/1855. Foreign priority was claimed to this Plant Breeder's Rights application on the U.S. Provisional Patent application for the instant plant.

### BACKGROUND OF THE INVENTION

The present invention relates to a new and distinct Lavandula plant, botanically known as Lavandula stoechas, commonly referred to as Lavender and hereinafter referred to by the name ‘DC014455LS’.

The new Lavandula plant is a product of a planned breeding program conducted by the Inventor in Comines, Belgium. The objective of the breeding program was to develop new Lavandula plants with good low temperature performance, attractive plant form and flower coloration and tolerance to pathogens.

The new Lavandula plant originated from an open-pollination during the summer of 2017 of Lavandula stoechas ‘LABZ0004’, disclosed in U.S. Plant Pat. No. 27,493, as the female, or seed, parent with an unknown proprietary selection of Lavandula stoechas as the male, or pollen, parent. The new Lavandula plant was discovered and selected by the Inventor as a single flowering plant from within the resultant progeny of the stated open-pollination in a controlled environment in Comines, Belgium in August, 2018.

Asexual reproduction of the new Lavandula plant by terminal cuttings in a controlled greenhouse environment in Comines, Belgium since September, 2018 has shown that the unique features of this new Lavandula plant are stable and reproduced true to type in successive generations.

### SUMMARY OF THE INVENTION

Plants of the new Lavandula have not been observed under all possible combinations of environmental conditions and cultural practices. The phenotype may vary somewhat with variations in environmental conditions such as temperature and light intensity, without, however, any variance in genotype.

The following traits have been repeatedly observed and are determined to be the unique characteristics of the new Lavandula plant. These characteristics in combination distinguish ‘DC014455LS’ as a new and distinct Lavandula plant:
1. Compact, upright and uniformly rounded plant habit.
2. Moderately vigorous growth habit.
3. Freely branching growth habit.
4. Early and freely flowering habit.
5. Dark purple-colored flowers and violet-colored terminal flower bracts positioned on strong and erect peduncles.
6. Long flowering period.
7. Suitable for low temperature production during the winter and early spring.
8. Good garden performance.

Plants of the new Lavandula differ primarily from plants of the female parent, ‘LABZ0004’, primarily in the following characteristics:
1. Plants of the new Lavandula are not as compact as plants of ‘LABZ0004’.
2. Lateral branches of plants of the new Lavandula are longer than lateral branches of plants of ‘LABZ0004’.
3. Inflorescences of plants of the new Lavandula are taller and narrower than inflorescences of plants of ‘LABZ0004’.
4. Terminal flower bracts of inflorescences of plants of the new Lavandula are narrower than terminal flower bracts of inflorescences of plants of ‘LABZ0004’.
5. Peduncles of plants of the new Lavandula are longer than peduncles of plants of ‘LABZ0004’.

Plants of the new Lavandula can be compared to plants of the Lavandula stoechas ‘LABZ0005’, disclosed in U.S. Plant Pat. No. 27,398. In side-by-side comparisons, plants of the new Lavandula differ primarily from plants of ‘LABZ0005’ in the following characteristics:
1. Plants of the new Lavandula are more compact than and not as vigorous as plants of ‘LABZ0005’.
2. Plants of the new Lavandula are more upright than plants of ‘LABZ0005’.
3. Inflorescences of plants of the new Lavandula are shorter than inflorescences of plants of ‘LABZ0005’.
4. Peduncles of plants of the new Lavandula are shorter than peduncles of plants of ‘LABZ0005’.

### BRIEF DESCRIPTION OF THE PHOTOGRAPHS

The accompanying photographs illustrate the overall appearance of the new Lavandula plant showing the colors as true as it is reasonably possible to obtain in colored reproductions of this type. Colors in the photographs may differ slightly from the color values cited in the detailed botanical description which accurately describe the colors of the new Lavandula plant.

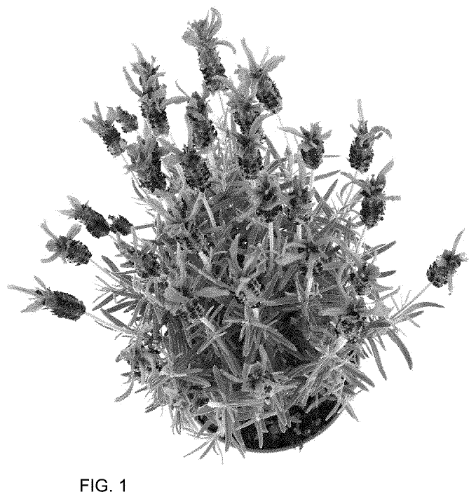

The photograph on the first sheet (FIG. 1) is a side perspective view of a typical flowering plant of ‘DC014455LS’ grown in a container.

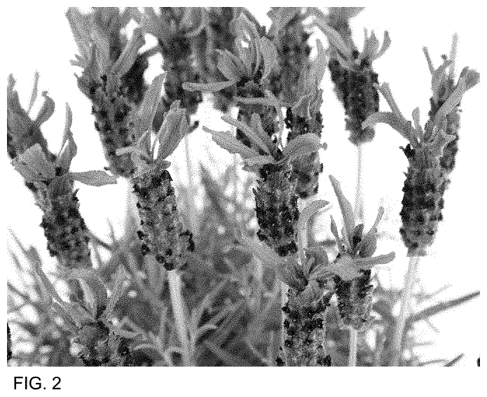

The photograph on the second sheet (FIG. 2) is a close up view of a typical flowering plant of ‘DC014455LS’.

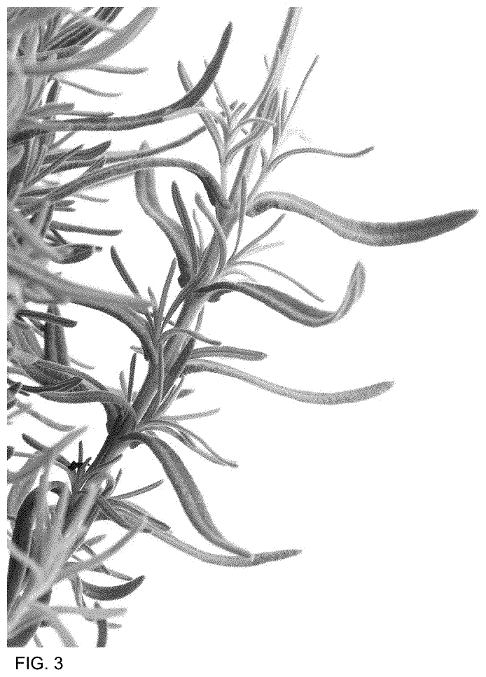

The photograph on the third sheet (FIG. 3) is a close up view of a typical stein and leaves of ‘DC014455LS’.

### DETAILED BOTANICAL DESCRIPTION

Plants used in the aforementioned photographs and following detailed description were grown during the early spring in a glass-covered greenhouse and finished in an outdoor nursery in Comines, Belgium. Plants were pinched three times and were grown under cultural practices typical of commercial Lavandula production. Single plants used for the photographs were grown in 10.5-cm containers and were 18 weeks old when the photographs were taken. Plants used for the description were grown with three plants in 24-cm containers and were 20 weeks old when the description was taken. During the production of the plants, day temperatures ranged from 20° C. to 30° C. and night temperatures ranged from 10° C. to 15° C. In the following description, color references are made to The Royal Horticultural Society Colour Chart, 2015 Edition, except where general terms of ordinary dictionary significance are used.

Botanical classification: Lavandula stoechas ‘DC014455LS’.
Parentage:
   Female, or seed, parent.—Lavandula stoechas ‘LABZ0004’, disclosed in U.S. Plant Pat. No. 27,493.
   Male, or pollen, parent.—Unknown proprietary selection of Lavandula stoechas, not patented.
Propagation:
   Type.—Terminal vegetative cuttings.
   Time to initiate roots, winter.—About 15 to 20 days at temperatures about 20° C.
   Time to produce a rooted young plant, winter.—About six weeks at temperatures about 20° C.
   Root description.—Fine to medium in thickness, fibrous; initially white in color (close to 158D) and becoming more brown with development; actual color of the roots is dependent on substrate composition, water quality, fertilizer type and formulation.
   Rooting habit.—Freely branching; medium density.
Plant description:
   Plant and growth habit.—Herbaceous perennial; compact, upright and uniformly rounded plant habit; roughly globular in overall plant shape; freely branching habit, dense and bushy appearance; moderately vigorous growth habit; moderate growth rate; flowers arranged in verticillasters on crowded spikes with showy terminal flower bracts.
   Plant height, soil level to top of foliar plane.—About 15 cm.
   Plant height, soil level to top of floral plane.—About 22 cm to 23 cm.
   Plant width.—About 19 cm to 22 cm.
   Lateral branch description.—Quantity per plant: About six to eight primary lateral branches per plant; each primary lateral branch with about four to six secondary lateral branches; pinching enhances branching potential. Length: About 18 cm to 21 cm. Diameter: About 2 mm. Internode length: About 1.3 cm to 2 cm. Strength: Strong. Aspect: Mostly upright. Texture and luster: Tomentose; matte. Color, when developing: Close to 138C; at the internodes, close to 138D. Color, developed: Close to 138D; proximally, variably tinged with close to 180D.
   Leaf description.—Arrangement: Opposite, simple; sessile. Length: About 3.7 cm to 4.3 cm. Width: About 3.5 mm to 5 mm. Shape: Ligulate. Apex: Acute. Base: Cuneate. Margin: Entire; slightly revolute; not lobed; proximally, slightly undulate. Texture and luster, upper surface: Pubescent; slightly rugose; matte. Texture and luster, lower surface: Pubescent; smooth to slightly rugose; matte. Fragrance: Strongly aromatic, pungent. Venation pattern: Pinnate; midrib prominent on lower surface. Color: Developing leaves, upper surface: Close to N138C. Developing leaves, lower surface: Close to 191B to 191C. Fully expanded leaves, upper surface: Close to 138B; venation, close to 138B. Fully expanded leaves, lower surface: Close to 146B or 147B; venation, close to 147B to 147C.
Flower description:
   Flower type, arrangement and habit.—Small single flowers arranged in compact verticillasters on crowded terminal spikes; freely flowering habit, about 68 to 87 flowers developing per inflorescence and about 30 to 35 inflorescences per plant; flowers with two-lobed upper lip and three-lobed lower lip; flowers face mostly outwardly to slanting upwardly on the spike; inflorescences with showy terminal flower bracts.
   Natural flowering season.—Plants begin flowering in April to May and in the garden, flower continuous through October in Belgium.
   Flower longevity on the plant.—Inflorescences last about four weeks on the plant; individual flowers last about five days on the plant; flowers not persistent.
   Fragrance.—None detected.
   Flower buds.—Length: About 3 mm. Diameter: About 1 mm to 2 mm. Shape: Oblong. Texture and luster: Smooth, glabrous; matte to slightly glossy. Color: Close to N89A.
   Inflorescence size.—Height, excluding terminal flower bracts: About 3 cm to 4.5 cm. Diameter, below flower bracts: About 1 cm to 1.4 cm.
   Flower size.—Diameter: About 3 mm to 4 mm. Depth (height): About 3 mm to 5 mm. Throat diameter: About 1 mm to 2 mm. Tube length: About 2 mm to 3 mm. Tube diameter: About 1 mm.
   Petals.—Quantity and arrangement: Upper lip, two-lobed and lower lip, three-lobed. Length, upper and lower lips: About 2 mm. Width, upper and lower lips: About 1 mm to 1.5 mm. Shape, upper lip: Roughly spatulate. Shape, lower lip: Tri-lobed. Apex, upper and lower lips: Obtuse. Margin, upper and lower lips: Entire; slightly undulate. Texture and luster, upper and lower lips, upper and lower surfaces: Smooth, glabrous; matte. Color: When opening, upper and lower surfaces: Close to N92A and N92D. Fully opened, upper and lower surfaces: Close to 83A; venation, close to 83A; color does not change with subsequent development. Throat: Close to 83D; venation, close to 83D. Tube: Close to N92D; venation, close to N92D.
   Terminal flower bracts.—Quantity and arrangement: About six to eight in one to two whorls slanting upright at inflorescence apex. Length: About 2.3 cm to 2.8 cm. Width: About 8 mm to 12 mm. Shape: Oblanceolate. Apex: Obtuse to cuspidate. Base: Attenuate. Margin: Entire; moderately undulate to occasionally somewhat twisted. Texture and luster, upper surface: Pubescent with prominent venation; slightly glossy. Texture and luster, lower surface: Pubescent with prominent venation; matte. Color: When developing, upper surface: Close to N81A to N81B. When developing, lower surface: Close to N81B. Fully developed, upper surface: Close to 86C; venation, close to N77C to N77D. Fully developed, lower surface: Close to N81B; venation, close to N77C to N77D.
   Basal flower bracts.—Quantity and arrangement: Each group of flowers is subtended by a single basal flower bract. Length: About 1 cm to 1.2 cm. Width: About 6 mm to 8 mm. Shape: Cordate or ovate. Apex: Acuminate. Base: Attenuate or cuneate. Margin: Irregularly dentate. Texture and luster, upper surface: Rugose, tomentose especially along the margins; matte. Texture and luster, lower surface: Smooth, glabrous; matte. Color, upper surface: Close to 177D; proximally, close to 195A; venation, close to N200A to lighter than N200A. Color, lower surface: Close to N77C to N77D; venation, close to 191A.
   Sepals.—Quantity and arrangement: Five, fused into a campanulate tube. Calyx length: About 5 mm to 6 mm. Calyx diameter: About 1.5 mm. Length: About 5.5 mm. Width: Less than 1 mm. Shape: Lanceolate. Apex: Acute. Margin: Entire. Texture and luster, upper surface: Tomentose; matte. Texture and luster, lower surface: Smooth, glabrous; matte. Color: When opening, upper and lower surfaces: Close to 144B. Fully opened, upper and lower surfaces: Close to 144B; venation, close to 144B.
   Peduncles.—Length: About 6 cm to 8 cm. Diameter: About 2 mm to 2.5 mm. Aspect: Mostly upright. Strength: Strong. Texture and luster: Densely tomentose; matte to slightly glossy. Color: Close to 191B; at the ridges, close to 194D.
   Reproductive organs.—Stamens: Quantity per flower: Four. Filament length: About 2 mm. Filament color: Whitish. Anther shape: Reniform; dorsifixed. Anther size: About 0.5 mm by 0.5 mm. Pollen amount: Scarce. Pollen color: Close to 13B. Pistils: Quantity per flower: One. Pistil length: About 4 mm to 5 mm. Stigma shape: Bi-lobed. Stigma diameter: About 0.5 mm. Stigma color: Light violet. Style length: About 4 mm. Style color: Pale yellow green. Ovary color: Yellow green.
   Seeds and fruits.—To date, seed and fruit production has not been observed on plants of the new Lavandula.
Pathogen & pest resistance: Plants of the new Lavandula have been observed to tolerate Botrytis Mold (Botrytis cinerea). To date, plants of the new Lavandula have not been noted to be resistant to pests and other pathogens common to Lavandula plants.
Garden performance: Plants of the new Lavandula have exhibited good tolerance to rain and wind and have been observed to tolerate high temperatures about 40° C. and to be suitable for USDA Hardiness Zones 8 to 10.

It is claimed:

1. A new and distinct Lavandula plant named ‘DC014455LS’ as illustrated and described.
